(19) United States
(12) Patent Application Publication
(10) Pub. No.: US 2021/0315898 A1
(43) Pub. Date: Oct. 14, 2021

(54) COMBINATION THERAPY FOR TREATING CANCER

(71) Applicant: AstraZeneca AB, Södertäje (SE)

(72) Inventor: Wolfram BRUGGER, Cambridge (GB)

(21) Appl. No.: 17/263,915

(22) PCT Filed: Jul. 26, 2019
(86) PCT No.: PCT/IB2019/056403

§ 371 Date: Jan. 28, 2021

Related U.S. Application Data
(60) Provisional application No. 62/711,753, filed on Jul. 30, 2018.
(60) Provisional application No. 62/727,152, filed on Sep. 5, 2018.

(51) Int. Cl. A61K 31/517; A61K 31/706; A61K 9/51; A61P 35/02

(52) U.S. Cl. CPC A61K 31/517; A61P 35/02; A61K 9/5153; A61K 31/706

(57) ABSTRACT

Disclosed are methods of treating cancer comprising administering to a subject in need thereof comprising administering to the subject an effective amount of a pharmaceutical composition comprising a plurality of AZD2811 nanoparticles and an effective amount of 5-azacitidine.

### RELATED APPLICATIONS

This application claims the benefit of priority under 35 U.S.C. § 119(e) to U.S. Provisional Patent Application No. 62/711,753, filed Jul. 30, 2018 and U.S. Provisional Patent Application No. 62/727,152, filed Sep. 5, 2018. The contents of the foregoing applications are hereby incorporated by reference in their entirety.

### BACKGROUND

While much progress has been made in the treatment of hematological malignancies, the many of these patients who have such cancers live with an incurable disease. Those patients suffering from acute myeloid leukemia (AML) have limited treatment options, and the five-year survival rate is approximately 25% with patients over 60 responding poorly to treatment, with a median survival of less than 12 months. Accordingly, it's important to continue to find new treatments for patients with incurable cancer.

### SUMMARY

In some embodiments, disclosed is a method of treating cancer comprising administering to a subject in need thereof an effective amount of a pharmaceutical composition comprising a plurality of AZD2811 nanoparticles and an effective amount of 5-azacytidine.

In some embodiments, disclosed is a pharmaceutical composition comprising a plurality of AZD2811 nanoparticles for use in the treatment of cancer, wherein said treatment comprises the separate, sequential or simultaneous administration of 5-azacitidine.

In some embodiments, disclosed is 5-azacitidine for use in the treatment of cancer, wherein said treatment comprises the separate, sequential or simultaneous administration of a pharmaceutical composition comprising a plurality of AZD2811 nanoparticles.

In some embodiments, disclosed is a kit comprising: a first pharmaceutical composition comprising a plurality of AZD2811 nanoparticles and a pharmaceutically acceptable carrier; and a second pharmaceutical composition comprising 5-azacitidine and a pharmaceutically acceptable carrier.

### BRIEF DESCRIPTIONS OF THE DRAWINGS

FIG. 1 illustrates the reduction in tumor volume over time of AZD2811 alone, 5-azacitidine alone, and the combination of AZD2811 and 5-azacitidine in a KG1a xenographic murine model of AML.

FIG. 2 illustrates the reduction in tumor volume over time of AZD2811 alone, 5-azacitidine alone, and the combination of AZD2811 and 5-azacitidine in a HL-60 xenographic murine model of AML.

FIG. 3 illustrates the survival proportions of mice treated with 5-azacitidine alone or with the combination of AZD2811 and 5-azacitidine

### DETAILED DESCRIPTION

In some embodiments, disclosed is a method of treating cancer comprising administering to a subject in need thereof an effective amount of a pharmaceutical composition comprising a plurality of AZD2811 nanoparticles and an effective amount of 5-azacitidine.

The language “AZD2811 nanoparticles” includes nanoparticles that comprise the Aurora kinase B inhibitor 2-(3-((7-(3-(ethyl(2-hydroxyethyl)amino)propoxy)quinazolin-4-yl)amino)-1H-pyrazol-5-yl)-N-(3-fluorophenyl)acetamide (also known as AZD1152 hqpa), about 7 to about 15 weight percent of pamoic acid, and a diblock poly(lactic) acid-poly(ethylene)glycol copolymer; wherein the diblock poly(lactic) acid-poly(ethylene)glycol copolymer has a poly(lactic acid) block having a number average molecular weight of about 16 kDa and a poly(ethylene)glycol block having a number average molecular weight of about 5 kDa; wherein the poly(ethylene)glycol block comprises about 10 to 30 weight percent of the therapeutic nanoparticle. Preparation of the AZD2811 nanoparticles is disclosed in International Application Publication No. WO2015/036792. In some embodiments, the AZD2811 nanoparticles are administered intravenously. In some embodiments, the AZD2811 nanoparticles are administered in a dose of up to about 600 mg of AZD2811 (for example, up to about 100 mg, up to about 200 mg, up to about 300 mg, up to about 400 mg, up to about 500 mg or up to about 600 mg AZD2811). In some embodiments, the AZD2811 nanoparticles will be administered intravenously over about 2 hours, over about 3 hours or over about 4 hours. In some embodiments, the AZD2811 nanoparticles are administered on day 1 and day 4 of a 28-day cycle.

The term “5-azacitidine” includes the compound of the structure:

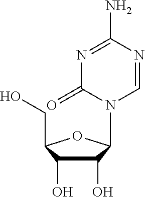

which is known as 4-amino-1-(β-D-ribofuranosyl)-1,3,5-triazin-2(1H)-one or ladakamycin. 5-Azacitadine is thought to have antineoplastic activity via two mechanisms—at low doses, by inhibiting of DNA methyltransferase, causing hypomethylation of DNA, and at high doses, by its direct cytotoxicity to abnormal hematopoietic cells in the bone marrow through its incorporation into DNA and RNA, resulting in cell death. In some embodiments, the method comprises administering to the subject a pharmaceutical composition comprising 5-azacitidine and mannitol. In some embodiments, the pharmaceutical composition comprises a 1:1 weight ratio of 5-azacitidine and mannitol (e.g., 100 mg each of 5-azacitidine and mannitol). In some embodiments, 5-azacitidine is administered subcutaneously. In some embodiments, 5-azacitidine is administered intravenously. In some embodiments, the 5-azacitidine is administered at 75 mg/m² daily for 7 days, followed by repeat cycles every four weeks, with an increase of 100 mg/m². In some embodiments, 5-azacytadine is administered at a 75 mg/m² dose on day 1 through day 7 of a 28-day cycle. In some embodiments, 5-azacytinde is administered at a 75 mg/m² dose on day 1 through day 5 and days 8 and 9 of a 28-day cycle.

The language “treat,” “treating” and “treatment” includes the reduction or inhibition of enzyme or protein activity related to Aurora kinase B, DNA methyltransferase or cancer in a subject, amelioration of one or more symptoms of cancer in a subject, or the slowing or delaying of progression of cancer in a subject. The language “treat,” “treating” and “treatment” also includes the reduction or inhibition of the growth of a tumor or proliferation of cancerous cells in a subject.

The language “inhibit,” “inhibition” or “inhibiting” includes a decrease in the baseline activity of a biological activity or process.

The term “cancer” includes but is not limited to hematological malignancies such as acute myeloid leukemia (AML), myelodysplastic syndrome (MDS) and chronic myelomonocytic leukemia (CMML). In some embodiments, the cancer includes cancers that are susceptible to treatment with Aurora kinase B inhibitors (e.g., AZD2811 nanoparticles). In some embodiments, the cancer includes cancers that are susceptible to treatment with DNA-methyltransferase inhibitors (e.g., 5-azacitidine).

The language “pharmaceutical composition” includes compositions comprising a plurality of AZD2811 nanoparticles and a pharmaceutically acceptable excipient, carrier or diluent. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. Pharmaceutical compositions may be in the form of a sterile injectable solution in one or more aqueous or non-aqueous non-toxic parenterally-acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The term “subject” includes warm-blooded mammals, for example, primates, dogs, cats, rabbits, rats, and mice. In some embodiments, the subject is a primate, for example, a human. In some embodiments, the subject is suffering from cancer. In some embodiments, the subject is suffering from relapsed AML. In some embodiments, the subject is suffering from relapsed high-risk MDS. In some embodiments, the subject is suffering from cancer and is treatment nave (e.g., has never received treatment for cancer). In some embodiments, the subject is in need of treatment (e.g., the subject would benefit biologically or medically from treatment). In some embodiments, the subject is pretreated with anti-nausea medication.

The language “effective amount” includes that amount of a pharmaceutical composition comprising AZD2811 nanoparticles and/or that amount of 5-azacitidine that will elicit a biological or medical response in a subject, for example, the reduction or inhibition of enzyme or protein activity related to Aurora kinase B, DNA-methyltransferase or cancer; amelioration of symptoms of cancer; or the slowing or delaying of progression of cancer. In some embodiments, the language “effective amount” includes the amount of a pharmaceutical composition comprising AZD2811 nanoparticles and/or 5-azacitidine, is effective to at least partially alleviate, inhibit, and/or ameliorate cancer or inhibit Aurora kinase B, DNA-methyltransferase, and/or reduce or inhibit the growth of a tumor or proliferation of cancerous cells in a subject.

In some embodiments, disclosed is a kit comprising: a first pharmaceutical composition comprising a plurality of AZD2811 nanoparticles and a pharmaceutically acceptable carrier; and a second pharmaceutical composition comprising 5-azacitidine and instructions for use.

### EXAMPLES

### Example 1

### Efficacy of AZD2811, a Selective AURKB Inhibitor, Combined with 5-azacytidine in Preclinical Models of Acute Myeloid Leukemia

KG1a: 2×10⁷ KG1a AML cells in 50% matrigel were implanted subcutaneously on the left flank of adult female SCID mice. Mice were randomised into groups of 12 and dosing was started for all drugs and all combinations at day 1 (D1) following implant. AZD2811 nanoparticles were dosed at once weekly with a 20-30 s intravenous infusion at 100 mg/kg (100 mg/kg was the maximum tolerated dose in combination 5-azacytidine at 1 mg/kg; 5-azacytidine was dosed twice-daily for three days by the intraperitoneal route, followed by 4 rest days of no dosing). All drugs were given for 3 weekly cycles. Tumors were measured twice weekly by single operators, and all dosing was performed by randomised cage to minimise systematic bias. Mice reached study surival endpoint when tumors reached 1.5 cm³.

HL-60: 1×10⁷ HL-60 AML cells in 50% matrigel were implanted subcutaneously on the left flank of adult female SCID mice. Mice were randomised into groups of 12 and dosing was started for all drugs and all combinations at day 7 respectively following implant. AZD2811 nanoparticles were dosed at once weekly with a 20-30 s intravenous infusion at either 25 mg/kg (100 mg/kg was the maximum tolerated dose in combination 5-azacytidine at 1 mg/kg; 5-azacytidine was dosed twice-daily for three days by the intraperitoneal route, followed by 4 rest days of no dosing). All drugs were given for 3 weekly cycles. Tumors were measured twice weekly by single operators, and all dosing was performed by randomised cage to minimise systematic bias. Mice reached study survival endpoint when tumors reached 1.5 cm³

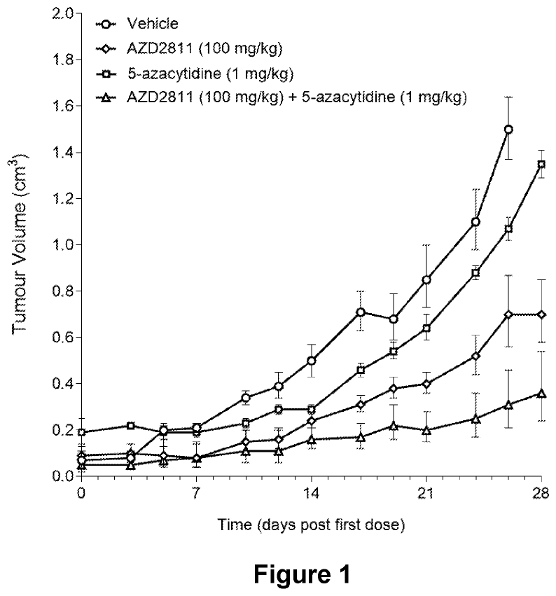

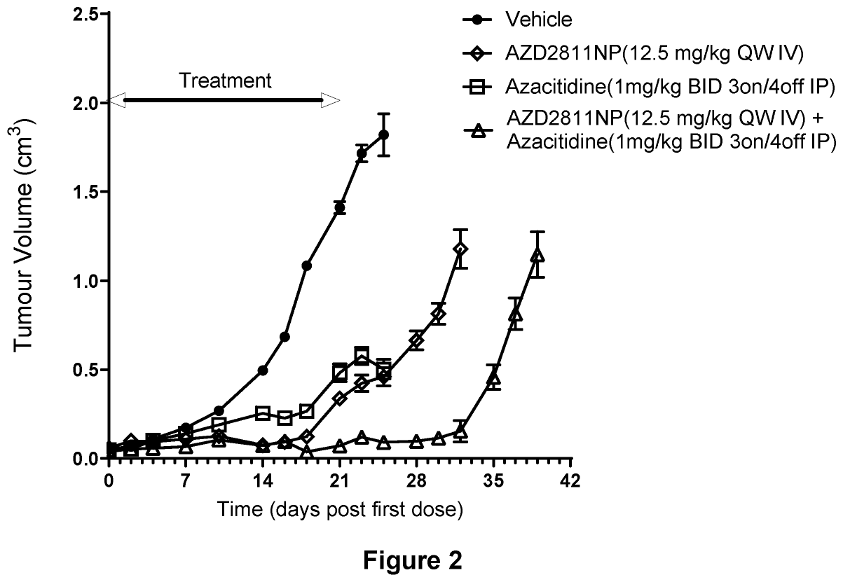

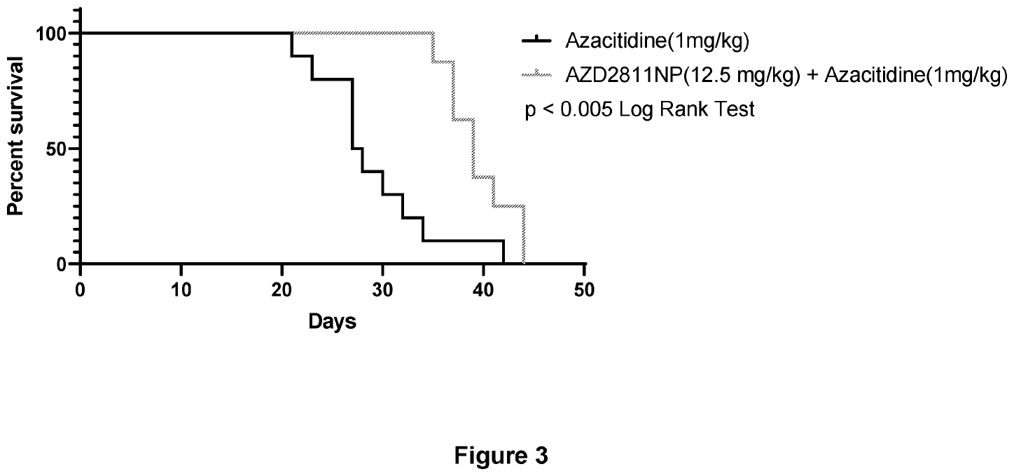

Results: As shown in FIGS. 1 and 2, both AZD2811 nanoparticles and 5-azacytidine monotherapy were modestly efficacious in the KG1a model with greater efficacy in the HL-60, and in both models, the combination of agents demonstrated markedly stronger efficacy. As shown in FIG. 3 the combination of AZD2811 and 5-azactidine gave a statistically significant survival benefit over 5-azacitidine alone in the HL-60 model (p<0.005; Log Rank test).

1. A method of treating cancer comprising administering to a subject in need thereof an effective amount of a pharmaceutical composition comprising a plurality of AZD2811 nanoparticles and an effective amount of 5-azacytidine.

2. The method of claim 1, wherein the method comprises administering the pharmaceutical composition comprising a plurality of AZD2811 nanoparticles sequentially, separately or simultaneously with 5-azacitidine.

3. The method of claim 1, wherein the cancer is a hematological malignancy.

4. The method of claim 3, wherein the hematological malignancy is selected from acute myeloid leukemia (AML), MDS and CMML.

5. A pharmaceutical composition comprising a plurality of AZD2811 nanoparticles for use in the treatment of cancer, wherein said treatment comprises the separate, sequential or simultaneous administration of 5-azacitidine.

6. 5-azacitidine for use in the treatment of cancer, wherein said treatment comprises the separate, sequential or simultaneous administration of a pharmaceutical composition comprising a plurality of AZD2811 nanoparticles.

7. The use of claim 5 or 6, wherein said cancer is a hematological cancer.

8. The use of claim 7, wherein the hematological cancer is selected from acute myeloid leukemia (AML), MDS and CMML.

9. A kit comprising:
a first pharmaceutical composition comprising a plurality of AZD2811 nanoparticles and a pharmaceutically acceptable carrier; and
a second pharmaceutical composition comprising 5-azacitidine and instructions for use.
